(19) United States
(12) Patent Application Publication
(10) Pub. No.: US 2015/0148621 A1
(43) Pub. Date: May 28, 2015

(54) METHODS AND SYSTEMS FOR CREATING A PREVENTATIVE CARE PLAN IN MENTAL ILLNESS TREATMENT

(71) Applicant: GRANT JOSEPH SIER, Barrington, RI (US)

(72) Inventor: GRANT JOSEPH SIER, Barrington, RI (US)

(21) Appl. No.: 14/550,986

(22) Filed: Nov. 22, 2014

Related U.S. Application Data
(60) Provisional application No. 61/907,481, filed on Nov. 22, 2013.

(51) Int. Cl. A61B 5/16; A61B 5/11; A61B 5/0205; A61B 7/00; A61B 5/01; A61B 5/00; A61B 5/0476

(52) U.S. Cl. CPC A61B 5/165; A61B 5/4806; A61B 5/7275; A61B 5/1112; A61B 5/0476; A61B 7/003; A61B 5/01; A61B 5/02055; A61B 5/4848; A61B 5/686; A61B 5/7246; A61B 5/0022; A61B 5/742; A61B 5/6898; A61B 5/6831; A61B 5/024
USPC 600/301; 600/300; 600/595; 600/544; 600/586; 600/549; 600/508; 600/547; 600/529

(57) ABSTRACT

Disclosed are methods, systems, and devices for generating sleep predicators to avoid preventable mental episodes from breaking through, thereby averting an irreversible brain damage to a patient, that may be caused by such future mental episodes. In one embodiment, the present invention is a method comprising: acquiring biomedical data from a patient by using a means of signal acquisition; forming a database of the biomedical records in which the database further comprises a sleep prediction algorithm, psychiatric records, and a statistical engine to compute the data and the psychiatric records using the algorithm; mapping the acquired data in reference to the sleep prediction algorithm; validating the sleep predictors; and providing an output of the sleep predicators to a patient or caretaker. The present invention can be used to treat mania, depression, bipolar disorder, schizophrenia, PTSD, anxiety, and other chronic mental health conditions.

### CROSS-REFERENCE TO RELATED APPLICATIONS

This application claims the benefit of a U.S. provisional application Ser. No. 61/907,481, entitled “Software for Correlating Physiological Processes with Bipolar Symptoms, Progression, and Therapeutic Side Effects,” filed on Nov. 22, 2013, which is hereby incorporated by reference in its entirety herein.

### FIELD OF THE INVENTION

Embodiments of the present invention broadly relate to medical devices. More particularly, embodiments of the present invention relate to medical devices in the prevention of mental illness.

### BACKGROUND OF THE INVENTION

The statements in this section may be necessary to a better understanding of the invention, but may or may not constitute prior art.

Patients with bipolar disease may improve their mental health condition by monitoring their own physiological and behavioral conditions (i.e., health data), and using their self-learned conditions in early diagnosis of future episodes and in aiding a psychiatrist's decision making process for an optimal treatment. A prior art method of monitoring mental health condition is often via a mood chart, or even via a mood chart related application in an electronic device.

A mood chart or a mood chart related application is often manually observed. Biomedical data used in such a chart is often inadequate. Such inadequacy may be because of lack of time-referenced data, insufficiency of quality data, lack of appropriate allocation of time devotion in recording such data, and the high cost of data entry by a health professional. Due to the inadequate collection of data, patients with severe mental illness, such as in mania, depression, bipolar disorder, schizophrenia, PTSD and anxiety, often do not get an optimal treatment plan, thereby risking their future mental wellness.

However, because of the aforementioned drawbacks of using prior art mood chart, or mood chart related applications, a preventable mental episode actually breaks through, costing irreversible brain damage to a patient.

Therefore, there is a long-felt and unresolved need in preventing potentially preventable future mental disease episodes from breaking through, for which prior art mood charts and mood chart related applications are inadequate. It is against this background that various embodiments of the present invention were developed.

### BRIEF SUMMARY OF THE INVENTION

The inventor of the present invention has created methods and systems for generating sleep predicators to avoid preventable mental episodes in the future from breaking through, thereby averting irreversible brain damage to a patient, that may be caused by such future mental episodes.

An unsolved problem of efficiently detecting future mental episodes and prescribing a preventative action plan early on, has been resolved by the present invention.

More precisely, in one embodiment, the present invention is a method of creating a plurality of sleep predicators of a patient, said plurality of sleep predicators predicting a serious mood episode of said patient's chronic mental illness, the method comprising the steps of, acquiring a physiological data and a behavioral data of said patient by using a means of signal acquisition, wherein said means of signal acquisition is selected from the group consisting of a transducer, a biomedical sensor, a surgically implanted sensor, a global positioning device, and a manually entered input; forming a database of psychiatric records by recording said data in said database, wherein said database is coupled with a statistical engine having a sleep prediction algorithm, said engine adapted to compute sleep predicators, wherein said engine statistically computes a plurality of psychiatric records and said data, and wherein said plurality of psychiatric records are acquired via at least one of public medical records and private medical records; mapping said acquired data in reference to said sleep prediction algorithm, in which the step of mapping comprises a statistical computation of said data, wherein said sleep prediction algorithm automatically adjusts based on an acquisition of a newer physiological data or a newer behavioral data, and wherein said sleep prediction algorithm achieves a statistical accuracy in sleep prediction by an iterative adjustment of said algorithm, wherein said iterative adjustment is based on said acquisition of said newer physiological or said newer behavioral data; validating said sleep predictors from said sleep prediction algorithm by evaluating said physiological data or said behavioral data in relation to said sleep predictors, thereby forecasting that a sleep pattern in said patient is about to change; and providing an output comprising said plurality of sleep predicators, wherein said output is displayed via a monitoring device, wherein said chronic mental illness is selected from the group consisting of manic episode, depressive episode, bipolar disorder episode, schizophrenic symptomatology, PTSD episode, and anxiety symptoms, and wherein said statistical computation comprises finding a first correlation between said acquired data and an illness symptom of said mental illness, wherein said illness symptom is statistically confirmed for accuracy by a first confirmation through said psychiatric records, thereby creating a therapeutic plan or a sleep remedy plan to prevent a further irreparable damage to said patient's mental state.

In another embodiment, the present invention is a monitoring device for monitoring a plurality of sleep predicators of a patient, said plurality of sleep predicators predicting a serious mood episode of said patient's chronic mental illness, the device capable of performing the steps taught in the above paragraph.

In another embodiment, the present invention is a system for creating a plurality of sleep predicators of a patient, said plurality of sleep predicators predicting a serious mood episode of said patient's chronic mental illness, in a client-server environment, the system comprising, a means of biomedical signal acquisition; a monitoring device having a processor, a first memory, and a display; a server having a second memory; a database of psychiatric records linked to said server; a communications-link between said monitoring device and said server; and a plurality of computer codes embodied on said first memory and on said second memory, said plurality of computer codes which when executed, causes said means of biomedical signal acquisition, said device, and said server to respectively execute a process comprising the aforementioned steps.

Other embodiments of the present invention will be apparent from the specification and diagrams as disclosed below.

### BRIEF DESCRIPTION OF THE DRAWINGS

Embodiments of the present invention described herein are exemplary, and not restrictive. Every figure or drawing should be read in accordance to an embodiment of the present invention, but not as the invention as a whole. Embodiments will now be described, by a way of examples, with reference to the accompanying drawings, in which:

FIG. 1 is a block diagram showing a data matrix that is used in some embodiments of the present invention.

FIG. 2 is a block diagram showing an exemplary wearable device according to one embodiment of the present invention.

FIG. 3 is a block diagram showing a recordation of EEG signal of a patient with mental illness according to one embodiment of the present invention.

FIG. 4 is a flow chart showing exemplary steps for generating sleep predictors, in accordance with one embodiment of the present invention.

FIG. 5 is a flow chart showing steps for improving a sleep prediction algorithm, in according with one embodiment of the present invention.

### DETAILED DESCRIPTION OF THE INVENTION

### Glossary Definitions

The following terms shall have the below definitions throughout this specification and claims. The terms may be used in the form of nouns, verbs or adjectives, within the scope of the definitions.
“Physiological Data” refers to data relevant to biology of human being. Such data comprise, but are not limited to, vital signs, EEG, vocal cord vibration, temperature, heart rate, muscle movement, EMG, conductivity, resistance, respiration rate, and UV light exposure.
“Behavioral Data” refers to the nature of a mental patient, such as, violent, screaming, disorganized, motivated, ability to control behavior, and the like. Behavioral data may overlap with mood related data, as a person with ordinary skill in the art knows that there may not be a clear separation between mood or behavior.
“Lifestyle Related Data” refers to habits or routines of daily life, such as, sleeping schedule, eating schedule, and the like.
“Mood Related Data” refers to emotional or mental status, such as, sadness, thoughts of suicide, anger, persistency, frustration, and the like.
“Transducer” is a device that converts a biomedical signal into an electrical signal or vice versa.
“Sensor” is a device that detects events and provides an output. A sensor may be electromechanical, optical, thermal, acoustic, or piezoelectric.
“Statistical Engine” is a computational or logical unit that can perform calculations based on data provided.
“Algorithm” refers to an effective method expressed as a finite list of well-defined instructions for calculating a function. Algorithm is a step-by-step procedure for calculations. Algorithms are used for calculation, data processing, and automated reasoning.
“Psychiatric Records” refer to any physiological, behavioral, lifestyle or mood related data of a patient, along with data relevant to medical or psychiatric care practice.
“Iterative” refers to continuous improvement by repeating data collection, modifying assumptions and correlations, and validating results.
“Sleep Analytics” refers to interpretation of sleep data for an intended medical or therapeutic purpose.
“Bipolar Disorder” refers to a manic-depressive illness, which is a brain disorder that causes unusual shifts in mood, energy, activity levels, and the ability to carry out day-to-day tasks.
“Depression” refers to severe and/or abnormally low mood symptoms that interfere with the ability to work, sleep, study, eat, and enjoy life.
“Schizophrenia” is a chronic, severe, and disabling brain disorder that has affected people throughout history. People with schizophrenia may hear voices other people don't hear. They may believe other people are reading their minds, controlling their thoughts, or plotting to harm them. This can terrify people with the illness and make them withdrawn or extremely agitated. People with schizophrenia may not make sense when they talk. They may sit for hours without moving or talking Sometimes people with schizophrenia seem perfectly fine until they talk about what they are really thinking.
“Mania” or “manic” refers to a mood of an abnormally elevated and/or aroused energy level.
“PTSD or Post-Traumatic Stress Disorder” refers to a mental health condition that is triggered by a terrifying event, either by experiencing it or by witnessing it. Symptoms of PTSD may include flashbacks, nightmares and severe anxiety, as well as uncontrollable thoughts about the event.
“Anxiety” refers to an unpleasant state of inner turmoil, often accompanied by nervous behavior, such as pacing back and forth, somatic complaints and rumination.
“Vital Signs” are used to measure the body's basic functions. These measurements are taken to help assess the general physical health of a person, give clues to possible diseases, and show progress toward recovery. The normal ranges for a person's vital signs vary with age, weight, gender, and overall health. Vital signs include your heart beat, breathing rate, temperature, and blood pressure.
“Biomarker” refers to a biological molecule found in blood, other body fluids, or tissues that is a sign of a normal or abnormal process, or of a condition or disease. A biomarker may be used to see how well the body responds to a treatment for a disease or condition. It is a measurable substance in an organism whose presence is indicative of some phenomenon such as disease, infection, or environmental exposure. Also called molecular marker and signature molecule.
“OCD or Obsessive-Compulsive Disorder” refers to an anxiety disorder characterized by intrusive thoughts that produce uneasiness, apprehension, fear or worry (obsessions), repetitive behaviors aimed at reducing the associated anxiety (compulsions), or a combination of such obsessions and compulsions.
“ADHD or Attention Deficit Hyperactivity Disorder” refers to one of the most common childhood disorders and can continue through adolescence and adulthood. ADHD symptoms include difficulty staying focused and paying attention, difficulty controlling behavior, and hyperactivity (over-activity).
“EKG or Electrocardiogram” refers to a test that checks for problems with the electrical activity of a heart.
“EEG or Electroencephalography” refers to a recording of electrical activity along a scalp. EEG measures voltage fluctuations resulting from ionic current flows within the neurons of the brain.
“EMG or Electromyography” refers to a technique for evaluating and recording the electrical activity produced by skeletal muscles.
The term “user” and “patient” are interchangeable, and mean the person with the mental illness that is utilizing the present invention.
The terms “psychiatrist,” “doctor,” “clinician,” “researcher,” and “medical professional” are interchangeable, and mean the medical caretaker of the patient.

### Overview

In the following description, for purposes of explanation, numerous specific details are set forth in order to provide a thorough understanding of the invention. It will be apparent, however, to one skilled in the art that the invention can be practiced without these specific details. In other instances, structures, devices, activities, and methods are shown using schematic, use case, and/or flow diagrams in order to avoid obscuring the invention. Although the following description contains many specifics for the purposes of illustration, anyone skilled in the art will appreciate that many variations and/or alterations to suggested details are within the scope of the present invention. Similarly, although many of the features of the present invention are described in terms of each other, or in conjunction with each other, one skilled in the art will appreciate that many of these features can be provided independently of other features. Accordingly, this description of the invention is set forth without any loss of generality to, and without imposing limitations upon, the invention.

Broadly, the present invention discloses methods, systems, and devices for generating sleep predicators to avoid preventable mental episodes from breaking out, thereby averting irreversible brain damage to a patient. Irreversible brain damage to a patient may be caused by such future mental episodes. Kindling is a result of multiple bipolar episodes, causing irreversible brain damage. It is an object of the present invention to avoid kindling by applying preventative measures. In one embodiment, the present invention is a method comprising, acquiring biomedical data from a patient by using a means of signal acquisition, forming a database of the biomedical records in which the database further comprises a sleep prediction algorithm, psychiatric records, and a statistical engine to compute the data and the psychiatric records using the algorithm, mapping the acquired data in reference to the sleep prediction algorithm, validating the sleep predictors, and providing an output of the sleep predicators.

Patients with bipolar disease may improve their mental health condition by monitoring their own physiological and behavioral conditions (i.e., health data), and using their self-learned conditions in early diagnosis of future episodes and in aiding a psychiatrist's decision making process for an optimal treatment. A prior art method of monitoring mental health condition is often via a mood chart, or even via a mood chart related application in an electronic device.

A mood chart or a mood chart related application is often manually observed. Biomedical data used in such a chart is often inadequate. Such inadequacy may be because of lack of time-referenced data, insufficiency of quality data, lack of time devotion in recording such data, and high cost of data entry by a health professional. Because of such inadequate data, patients of severe mental illness, such as depression, bipolar disorder, schizophrenia, PTSD and anxiety, do not get an optimal treatment plan, thereby risking their future mental disease state break throughs.

However, because of the aforementioned drawbacks of using prior art mood chart, or mood chart related applications, a preventable mental episode actually breaks through, costing irreversible brain damage to a patient.

Therefore, there is a long-felt and unsolved need in preventing preventable future mental episodes from clinically breaking through, for which prior art mood charts and mood chart related applications are inadequate.

Accordingly, the inventor of the present invention has created methods and systems for generating sleep predicators to avoid preventable mental episodes from breaking out, thereby averting an irreversible brain damage to a patient, that may be caused by such future mental episodes. An unsolved problem of efficiently detecting future mental episodes and prescribing a preventative plan early on, has been resolved by the present invention.

More precisely, in one embodiment, the present invention is a method of creating a plurality of sleep predicators of a patient, said plurality of sleep predicators predicting a serious mood episode of said patient's chronic mental illness, the method comprising the steps of, acquiring a physiological data and a behavioral data of said patient by using a means of signal acquisition, wherein said means of signal acquisition is selected from the group consisting of a transducer, a biomedical sensor, a surgically implanted sensor, a global positioning device, and a manually entered input; forming a database of psychiatric records by recording said data in said database, wherein said database is coupled with a statistical engine having a sleep prediction algorithm, said engine adapted to compute sleep predicators, wherein said engine statistically computes a plurality of psychiatric records and said data, and wherein said plurality of psychiatric records are acquired via at least one of public medical records and private medical records; mapping said acquired data in reference to said sleep prediction algorithm, in which the step of mapping comprises a statistical computation of said data, wherein said sleep prediction algorithm automatically adjusts based on an acquisition of a newer physiological data or a newer behavioral data, and wherein said sleep prediction algorithm achieves a statistical accuracy in sleep prediction by an iterative adjustment of said algorithm, wherein said iterative adjustment is based on said acquisition of said newer physiological or said newer behavioral data; validating said sleep predictors from said sleep prediction algorithm by evaluating said physiological data or said behavioral data in relation to said sleep predictors, thereby forecasting that a sleep pattern in said patient is about to change; and providing an output comprising said plurality of sleep predicators, wherein said output is displayed via a monitoring device, wherein said chronic mental illness is selected from the group consisting of a mania, a depression, a bipolar disorder, a schizophrenia, a PSDT and an anxiety, and wherein said statistical computation comprises finding a first correlation between said acquired data and an illness symptom of said mental illness, wherein said illness symptom is statistically confirmed for accuracy by a first confirmation through said psychiatric records, thereby creating a therapeutic plan or a sleep remedy plan to prevent a further irreparable damage to said patient's mental state.

In another embodiment, the present invention is a monitoring device for monitoring a plurality of sleep predicators of a patient, said plurality of sleep predicators predicting a serious mood episode of said patient's chronic mental illness, the device capable of performing the steps taught in the above paragraph.

In another embodiment, the present invention is a system for creating a plurality of sleep predicators of a patient, said plurality of sleep predicators predicting a serious mood episode of said patient's chronic mental illness, in a client-server environment, the system comprising, a means of biomedical signal acquisition; a monitoring device having a processor, a first memory, and a display; a server having a second memory; a database of psychiatric records linked to said server; a communications-link between said monitoring device and said server; and a plurality of computer codes embodied on said first memory and on said second memory, said plurality of computer codes which when executed, causes said means of biomedical signal acquisition, said device, and said server to respectively execute a process comprising the aforementioned steps.

### DESCRIPTION OF THE FIGURES

With reference to the figures, embodiments of the present invention are now described in detail.

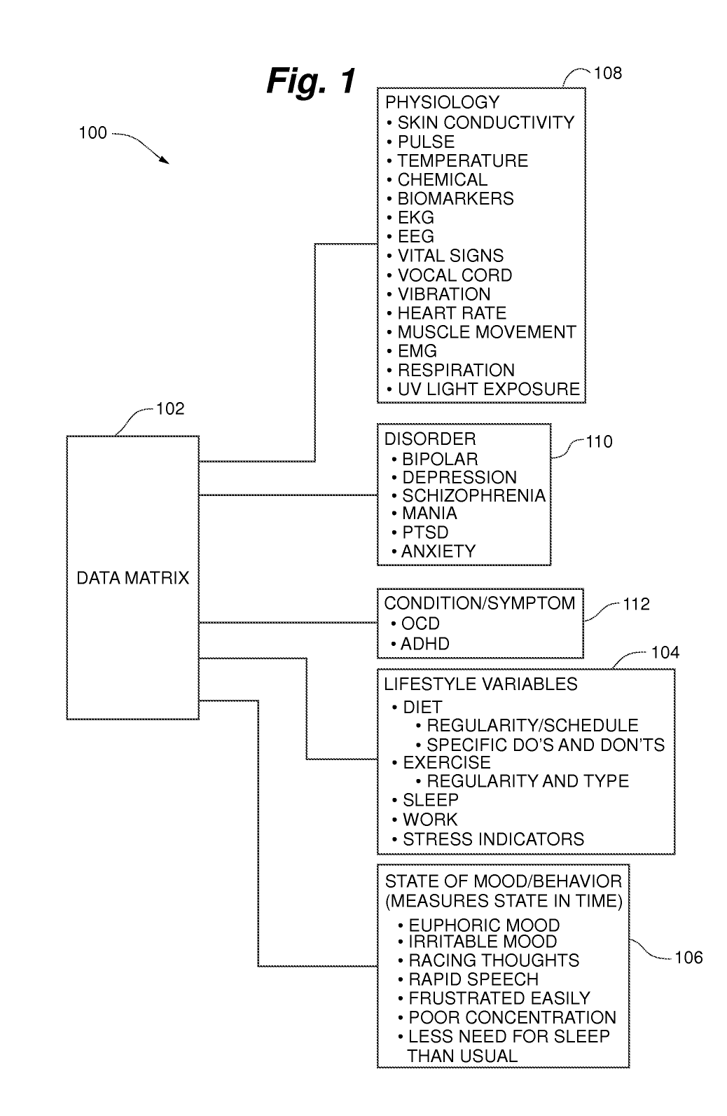

FIG. 1 is a diagram showing an embodiment 100 of data matrix that may be used by the present invention. The data matrix 102 comprise physiology 108, disorder 110, condition/symptom 112, lifestyle variables 104, and state of mood/behavior 106. The physiology 108 comprises data relating to skin conductivity, pulse, temperature, chemical exposure, biomarkers, EKG, EEG, vital signs, vocal cord vibration, heart rate, muscle movement, EMG, respiration, and UV light exposure. The disorder 110 comprises data relating to bipolar, depression, schizophrenia, mania, PTSD and anxiety. The condition/symptom 112 comprise data relating to OCD and ADHD. The lifestyle variables 104 comprise data relating to diet, exercise, sleep, work, and stress. A diet data may comprise regularity or schedule of diet. An exercise data may comprise regularity and type of exercise. The state of mood/behavior 108 measures a data at a state that may be referenced by time. Such mood/behavior data may comprise data relating to euphoric mood, irritable mood, racing thoughts, rapid speech, frustration, concentration, and need for sleep. A person with ordinary skill in the art of the present invention realizes that there may be other similar data that may be used in the data matrix. This data matrix 102 is not an exhaustive list, but just a showing of illustrative examples.

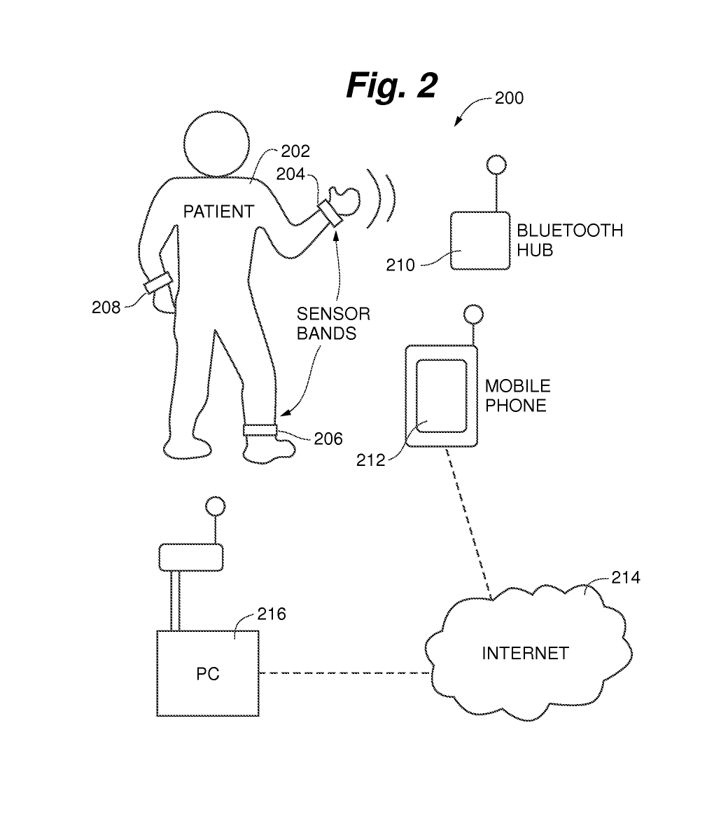

FIG. 2 is an embodiment 200 showing a wearable device of the present invention. A wearable device may be a sensor band that may be worn on hand or foot, or any other convenient part of body. A patient 202 may use hands to wear a first sensor band 204 and a second sensor band 208. The patient 202 may also use a foot to wear a third sensor band 206. The sensor bands may be adapted to communicate with a Bluetooth hub 210, a mobile phone 212, or a personal computer 216. The mobile phone 212 and the personal computer 216 are connected to the Internet 214. The Bluetooth hub 210 is adapted to receive data from the patient 202 and transmit the same data to either the mobile phone 212, or the personal computer 216.

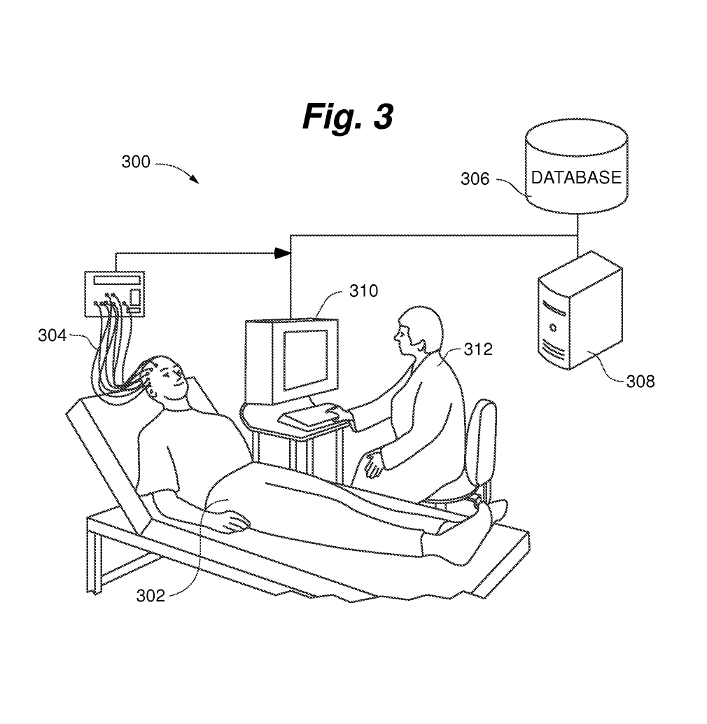

FIG. 3 is an embodiment 300 showing a patient who is recoding his EEG (Electroencephalography) signals to verify his symptoms of mental illness. A healthcare professional 312 uses a recording device 304 to record EEG signals of a mental patient 302. The EEG signals are recorded in the database 306. A server 308 performs necessary statistical analysis of the EEG signals and transmits an output to a PC monitor 310. The server 308 has a statistical engine to perform a statistical analysis of the EEG signals. A statistical analysis is performed by using a sleep prediction algorithm. The sleep prediction algorithm creates sleep predicators using biomedical signals, such as, EEG signals. The healthcare professional 312 studies the output using the PC monitor 310.

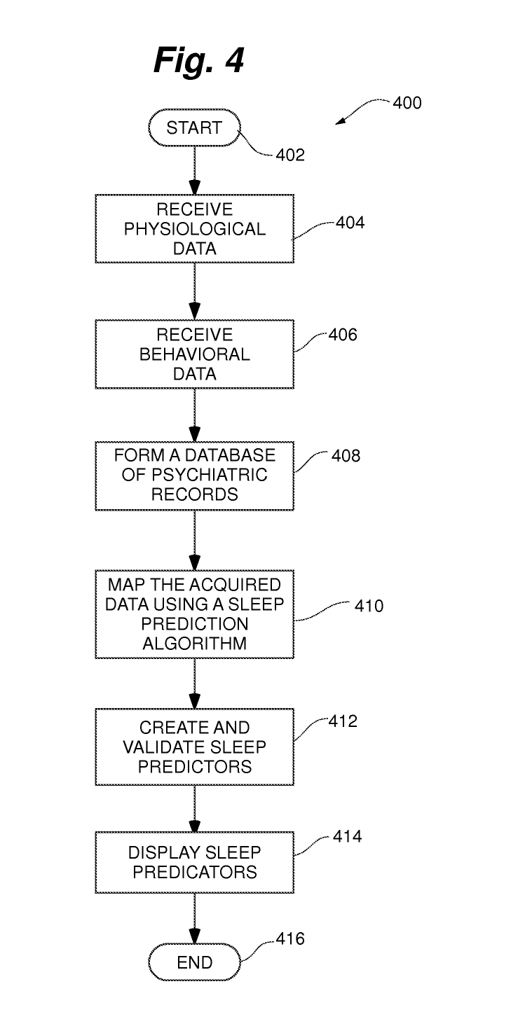

FIG. 4 is a flow diagram of an embodiment 400 showing a process of creating sleep predictors. The embodiment 400 uses hardware (not shown) comprising a means of signal acquisition (for example, a biomedical sensor), a monitoring device, a database and a server having a sleep prediction algorithm. At step 402, the process starts. A step 404, a means of signal acquisition receives physiological data from a mental patient. At step 406, the process receives behavioral data of the patient. Mood related data and/or lifestyle related data may also be added to the process. At step 408, the received physiological data and behavioral data, in addition to mood related data and/or lifestyle related data, are recorded in a database, thus forming a database of psychiatric records. This database may pull additional psychiatric records from relevant private and/or public medical records for data verification purpose or illness symptom confirmation purpose. At step 410, the process maps the received or acquired data using a sleep prediction algorithm. At step 412, the method creates and then validates sleep predicators using the sleep prediction algorithm. At step 414, the sleep predicators are sent as an output or result, and displayed via a monitoring device. The method ends at step 416.

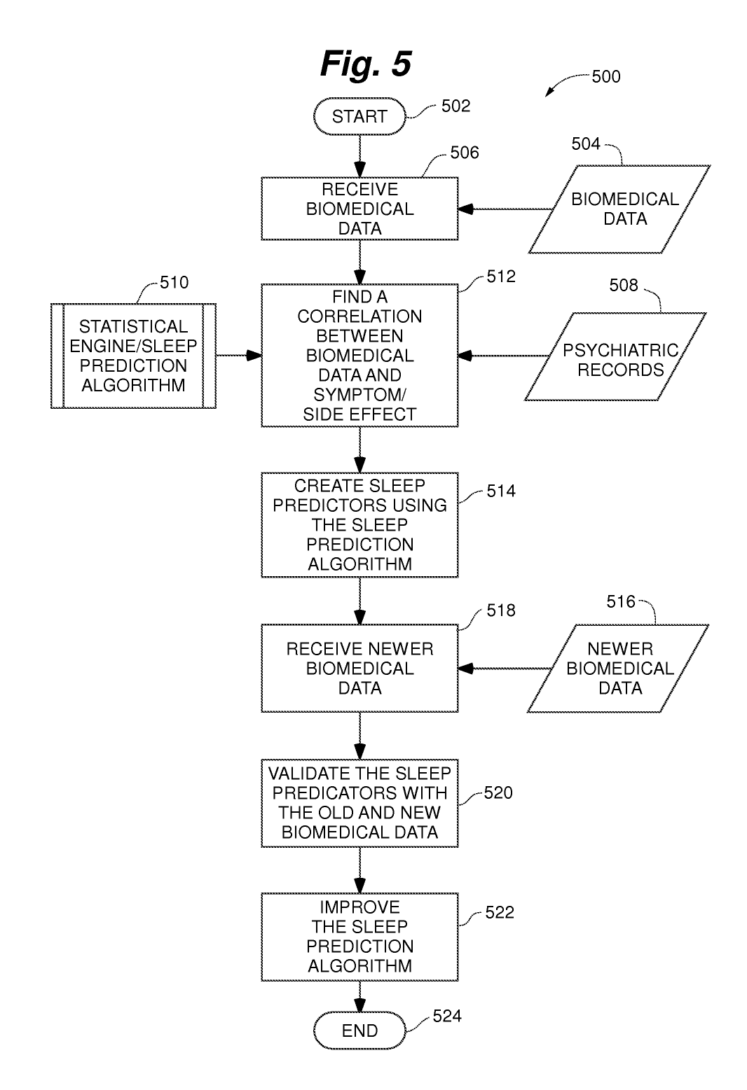

FIG. 5 is a flow diagram of an embodiment 500 showing a process of improving (self-improvement) of a sleep prediction algorithm of the present invention. The process starts at step 502. At step 504, a means of signal acquisition or a biomedical sensor acquires biomedical data of a mental patient. At step 506, the acquired data is received for processing by the process. Once the data is received, the data is stored in a database. The database comprises a statistical engine 510 that has a sleep prediction algorithm. The database also comprises psychiatric records 508 that may be obtained from public or private medical records. Using the medical records 508, the received biomedical data 504, and the sleep prediction algorithm 510, at step 512, the process finds a first correlation between the collected data and a symptom that is associated with a relevant mental illness. The process may also find a second correlation between the collected data and a side effect that is associated with a relevant mental illness. A side effect is often a therapeutic side effect of treatment of the mental illness, in which the therapeutic side effect is related to a drug regimen of the patient. The first correlation, with or without the aid of the second correlation, confirms the state of illness, and validates the illness, predicts side effects, learns the causes of the illness, and forecasts a preventative plan to avert a future mental episode or breakout. A preventative plan may comprise a sleep plan. A preventative plan comes with a warning that a sleep pattern of the patient is about to change. The acquired data when mapped by using the sleep prediction algorithm predicts that a sleep pattern is about to change. When sleep pattern changes, a state of mental illness is affected. Before the sleep pattern is changed, a healthcare professional, or even the patients themselves, can apply a relevant preventative plan to normalize a sleep pattern. At step 514, the process creates sleep predicators using the sleep prediction algorithm of the present invention. The sleep prediction algorithm is not a static or sealed algorithm, but an evolving or self-adjusting one. As step 516, the database of records grows by adding more or newer biomedical data. The process at step 518 receives the more and newer data. At step 520, the sleep prediction algorithm uses validation techniques using statistical means on the more and newer data. Correlations between biomedical data and illness symptoms (or side effects) are relevant in illness validation and in forecasting a preventative plan to avert a future mental episode or break out. The sleep prediction algorithm becomes statistically more accurate as there are newer findings or newer confirmations of mental illness through the acquired data and/or psychiatric records. As more correlations become relevant in illness prediction, the sleep prediction algorithm improves statistically or mathematically at step 522. The process ends at step 524.

### Exemplary Illustrative Embodiments of the Invention

A more restrictive or contextual language may be used in this section for illustration purposes only and for ease of understanding, but is not intended to limit the present invention or its uses to the examples described herein. The spirit of the present invention is reflected by the entirety of the specification, not just this section. The contextual language in this section may serve as basic ingredients or parameters to the sleep prediction algorithm in one embodiment of the present invention.

Bipolar disorder is the sixth leading cause of disability in the world. This disease affects approximately 5.7 million Americans. Bipolar disorder results in 9.2 years reduction in expected life span, and as many as one in five patients with bipolar disorder successfully completes suicide. This is a devastating disease that ruins the lives of patients and their loved ones.

Interestingly, bipolar disorder is a treatable illness. With very tight monitoring, good education and patient compliance, the disease can go into remission for years, decades or even for the remainder of a patient's life.

Bipolar episodes are initiated by sleep changes as well as perpetuated by these sleep changes. Sleep deprivation, specifically a decreased need for sleep, is one fundamental cause of mania. Sleep deprivation is both a cause and a consequence of mania. Self-reinforcing sleep loss perpetuates the manic state. An increased need for sleep and overwhelming fatigue despite adequate or even excessive sleep marks the beginning of a bipolar depressive episode. Weight gain and increased appetite (specifically a craving for carbohydrates due to a shortage of serotonin) often accompany these episodes, pointing to the metabolic physiological changes occurring in the brain and body as depression begins to manifest.

Bipolar episodes seem as if they come out of the blue to an outside observer. However, there are many prodromal changes that occur insidiously over time. Science has not established clear patterns for many of these psychological and physiological changes. The variable that we do know has a clear prodromal pattern is sleep.

At this point in time, sleep is the variable that will ultimately define a bipolar state. Sleep changes always precede serious mood changes. The time frame for each individual, however, is very different. That one common similarity, if properly monitored, can predict changes that once triggered cause a devastating condition where a patient's insight and/or self-control disappears and intervention becomes very difficult, and in some cases, impossible. Having solid data that accurately predicts an episode that is building up in a person allows intervention at a point where the patient still has insight and can be compliant with aggressive treatment strategies that successfully prevent the episode from ever occurring. Furthermore, kindling is a phenomenon that occurs in the brain with every bipolar episode. Basically, irreversible brain damage is done as a result of each episode. It takes quite a toll on the brain. Although humans are neuroplastic and have a lot of “play” in the brain to be able to tolerate occasional hits, multiple episodes on a long-term basis cause permanent brain damage on a large scale that becomes evident later in life. This is another very important motivation to prevent episodes from reoccurring.

The present invention will take mood charting into consideration. Mood charting has historically limited usefulness clinically due to multiple irregularities on the physician and patient ends. The most important physiological variable with known clinical utility is sleep, as we have discussed above. Collecting data at regular intervals and capturing it on a computer will allow for ease of compliance as well as seeing potential changes in psychological and physiological parameters from hour to hour, and not just day to day.

Over time, a database per patient and for the entire system (all patients in the database) will form. On the individual patient end, the particular nuances for each individual's disease will be elucidated and prevention interventions prior to an episode will be accurate and effective. On the macroscopic mental health end, new correlations will be elucidated between variables and with this new knowledge, further preventions will be able to be instituted on this larger scale.

There are many problems in mental health care today. Besides the impact that the shortage of psychiatrists has on mental health care in general, the majority of psychiatrists are unable to spend adequate time with their patients due to the severe demands and restrictions on mental health services by managed care companies. Most patients are seen for 10 to 15 minutes per visit. This is simply not enough time to get to know a patient, never mind enough time to learn the individual idiosyncrasies of that patient. This has led us to a culture in psychiatric care of using the limited timeframe available to simply try to put out fires. Fires can be difficult to put out. They often cause damage and devastation. This application is designed to prevent the consequences of the devastating fires. That changes everything if you are an individual with bipolar disorder.

Consumers who report high levels of satisfaction with their treatment and treatment provider have a much more positive outlook about their illness and their ability to cope with it. The present invention is designed to incorporate fastidious care from a compassionate psychiatrist working very closely with the individual patient using the system. The importance of this concept is astounding in terms of eradicating a potentially devastating disease state. What is really exciting is that bipolar patients on a macroscopic level will be able to shed their culturally imposed shame as they gain control over their brain difference.

Bipolar disorder is an important public health issue and is one of the most expensive mental diseases in terms of treatment and insurance purposes. Beyond psychiatry's clinical settings there is great need for complimentary tools such as mood charting. Daily mood charting often helps the patient recognize their conditions and improve their ability to cooperatively diagnose their symptoms alongside of their medications and environmental factors that come into play. Self-knowledge of these conditions in a patient's disorder could improve early diagnosis of new episodes and aid a psychiatrist's decision making process for the optimal possible treatment. However, there are many limitations that exist in conventional paper/pencil mood charting techniques. Traditionally, patients have used paper-based mood charts for self-diagnosis, but they come with the problems of data quality, insufficient compliance, high costs of data entry, and result in inadequate feedback for the patients and psychiatrists. Therefore, prior art mood charting is often inefficient. A study consisting of bipolar patients discovered that weekly clinician ratings captured only 31.4% of days of depression and 14.1% of days of mania by patients using pencil/paper mood charting. Many drawbacks reduce the success rate of paper/pencil mood charting. One problem is compliance, which could be improved upon by the availability of smart phone technology that can be used in a more natural environment. Also, smart phone applications provide superior graphical representations, which can be unique and insightful for each application individually.

However, there are still areas within smartphone applications that require improvements. For example, no time-reference is recorded when the patient fills out their scaled symptoms. Side-effects are not personalized specifically to the patient's drug regimen and are not scaled objectively. In addition to this, most smartphone mood charting applications only allow the patient to log one severity of a symptom per day. The nature of bipolar illness is that symptoms can be sporadic and change even by the hour in terms of severity. One more problem with paper/pencil mood charting, as well as smartphone applications, is that these formats make no attempts to create a database, which could be used by researchers. These are some of the dilemmas that exist under the traditional paper/pencil and smart-phone Apps for mood charting. The present invention resolves limitations of such smart phone applications. A smart phone is an exemplary device for monitoring. A monitoring device is not limited to smart phones.

The present invention may calculate and display diagnostic information, graphical and statistical, to the patient and/or psychiatrist via the screen of a mobile device. If certain combinations of symptoms or side-effects are detected, the software may send an automatic alert to the patient to seek immediate treatment. Continuous and precise results throughout cross-examinations in the database provides a system wherein query of the patient can be replaced by objective correlations, trends, and patterns.

In one example, a system, a device or a method described herein comprises an application configured for biomedical monitoring and an algorithm of self-adjusting mood charting, further comprising physiological, psychological, behavioral and mood-related variables. One embodiment is capable of running on a mobile device, such as a smart phone having a biomedical sensor or biomedical monitoring device. Data gathered by the mobile device and application from the sensor will be relayed to a central database for further analysis and study, via a wireless connection. The application will include a local database for storing an individual patient's symptoms and data. Continuous and precise results throughout cross-examinations in the database provides a system wherein a query of the patient can be replaced by objective correlations, trends and patterns.

It is envisioned that the application may be written in any programming language and designed to function on any mobile device hardware and operating system, such as GOOGLE ANDROID operating system and various smartphone hardware manufacturing partners, APPLE iOS and iPHONE, MICROSOFT and WINDOWS phones, BLACKBERRY, and the like.

Mobile devices or monitoring devices generally include a touch sensitive screen for input, with a limited or a few buttons. Such devices include a processor with one or more CPU cores, a volatile random access memory, a non-volatile flash memory, microphone and speakers, and one or more radios operating at various standard frequencies for wireless communications.

A monitoring device may be any sensor configured to collect and transmit a biomedical reading from a patient. A biomedical sensor can detect, quantify or measure the constant changes of physiological processes by monitoring the human anatomy, which can be used to measure chronic symptoms (in this case bipolar disease). The sensor may be configured to sense electrochemical, optical, thermal, acoustic, or piezoelectric inputs, and the like. The sensor may be used to collect measurements such as vital signs, EEG, vocal chord vibration analysis, skin temperature, heart rate, muscle movements, EMG, conductivity or resistance, respiration, and UV light exposure. The sensor may be wearable or may be attached to the surface of a patient's skin, or even may be surgically implantable. The sensor may be capable of wireless communications in order to transmit or be interrogated for readings collected from the patient. Such sensors may be low powered, miniaturized, or a system-on-a-chip design, in order to minimize cost and inconvenience to the patient, although other design choice may be used as seen fit by a person of ordinary skill in the art.

There is much debate in the psychiatric community regarding what guidelines and descriptions should be applied to aspects of bipolar disease. In this disclosure, the terms “state” and “symptom” are interchangeably applied when referring to aspects of bipolar disease. Mania and depression are states of bipolar disease, not symptoms. To capture overall mania or depression, a patient may need to experience three or more subcategories of symptoms that are present during each state. Throughout each state, one or more episodes occur intermittently and loosely follow a bell curve trend in terms of severity. Most bipolar patients are considered to be semi-symptomatic. This means that the states of their disease follow a predictable patterns that progress in predictable sequence such as from baseline, to mania, followed by depression. There may be many other possible combinations. Once this sequence starts over again, the patient is considered to complete one full cycle. Cycles can last anywhere from one to several years. Mood charting has been traditionally used to document a patient's cycle and symptoms within each state of the cycle. This information is usually scaled on a number line for severity of symptoms and is represented graphically for interpretive analysis.

In another embodiment, the present invention has two main characteristics that work off of each other in cohort: biomedical monitoring and an algorithm of self-adjusting mood charting, along with psychological and life style variables. The mood charting and other psychological variables rated by the patient may reflect more subjective aspects of the illness. The monitored biometric recordings made and documented by biomedical sensors may reflect more objective aspects of the illness. When taken together, they represent a more complete picture of the patient's situation and well-being.

In another embodiment, the present invention will contribute to identifying correlations between physiology and targeted bipolar symptoms as well as side effects of therapeutic medications that can be found through monitored processes. Since many unique combinations of symptoms/side effects may be present for each patient, cross examination between patients in the database will prove to be relevant in targeting specific symptom(s) and their significance. It is important that these monitored readings are invisible to the user, since they may bias the input of the mood charting. However, the monitored readings should be available to psychiatrist/researchers as an adjunct to diagnosis along with other clinical observations. A software application (or a mobile “App”) may be made, which may even be tailored by a psychiatrist to decide what symptoms are relevant to monitor, and to avoid false positive and false negative readings. One aspect of this application or App is that biomedical monitoring and a self-adjusting mood charting (algorithm) and other psychological variables can be tailored to one another in a unique way to accomplish the overall goal of this invention in the study/research phase. One goal of this invention also remains relevant in the clinical treatment settings.

According to an aspect of the present invention, a new algorithm for a modified or self-adjusting mood charting is necessary to carry over for compliance in the post-study clinical setting. In one aspect, one embodiment mimics techniques that may be used by psychiatrists. In another aspect, one embodiment is a passive monitoring combined with subjective input from the users regarding their current mood. The inputs for the algorithm for a modified or self-adjusting mood charting is crucial to developing correlations amongst one or more symptoms/side effects to patterns in the database. In another embodiment, this invention uses the mood charting input to provide descriptions for the monitored biometric readings. By tracking the two alongside of one another and applying algorithmic mathematical (computational) programming, the invention attempts to identify correlations between the two, thus revealing connections between bipolar symptoms and corresponding physiology. The user's input is transmitted to a central database. Using the database in the study phase and moving forward, pattern recognition and algorithms may be applied to identify and document correlations and trends between and among patients, with targeted symptoms and side effects. Additionally, unsupervised analysis may reveal previously unknown targets. In a clinical stage, this invention may use these connections as guidelines for individual cases in documenting trends and correlations between outputs. There are many aspects and guidelines that are incorporated into the self-adjusting mood charting algorithm to achieve the overall desired effect of the invention, such as the following.

Calculating the medians of correlations in the database to use as reference for comparisons in individual cases. Once the patient has cycled multiple times, then the trends and correlations from the previous cycles become the benchmarks for comparison of new episodes.

For organizational purposes in the database, all like factors should be categorized together. This will prove to be effective for later analysis (e.g. similar medication combinations (including dosage)).

The self-adjusting mood charting algorithm includes several unique features, making it different than prior art mood charting applications.

First, side-effects and symptoms are captured. Side effects are tailored to each patient according to their drug regimen. Identifying these side effects is a dual effort on part of the patient and the psychiatrist, since a psychiatrist is trained to diagnose certain side effects that patients would otherwise be unable to self-diagnose. Some side effects like increased heart rate are objectively measured, so the baseline database should be used as a benchmark to capture severity of the side effect. However, subjective input from mood charting should still be used to identify certain side effects, especially in-between clinical visits when observations are not possible and documentation will be useful in retrospective tracking and analysis.

Second, symptoms/side effects under specific periods of time throughout a day (not simply one log per day) are captured. Even for those experiencing only a few symptoms a day, the severity of the specified symptom is likely to vary throughout the day and so will the monitored output respectively. This allows a timeline to be tracked for the interpretation of the monitored output.

Another aspect of the invention involves an interactive algorithm of mood charting and other psychological and life style factor ratings for personalized input and output. This allows the invention to interact with the user and intelligently request patients to log symptoms/side effects during time periods of interest, when the monitored output seems to follow a predicted trend/correlation previously discovered. These trends are identified in the study phase of the application and are later applied individually to the user.

In one embodiment, the goal of the input is to create more objective measurements by correlating mood assessment to physiology. In another embodiment, the goal is to capture self-adjusting mood charting and other psychological and lifestyle variables. In practice, the patient tells the App what input to login based on self-diagnosis/scaling of symptoms and side-effects. This input will in turn provide the program with information to create correlations alongside of physiology by applying pattern recognition to the two inputs. So these initial correlations are dependent upon patient input/self-diagnosis in order to create correlations based upon pattern recognition.

Next, pattern recognition may be applied to passive monitoring of physiological input, in effect acting as a detector for correlations. In order to avoid false positives the App asks the patient if these correlations are accurate.

One object of this invention is to have frequent recognition of patterns. So when these recognitions/patterns are made, the App will already know the input from which the patterns were correlated. Thus the pattern can provide an automatically updated input into the self-adjusting mood charting algorithm, simply by detecting the patterns in physiology. A simple multiple choice questionnaire can make sure that the updated input into the self-adjusting mood charting algorithm is valid of the patient's condition. This input should reflect the symptoms/side effects (including severity and weight) based on the pattern detected in the monitored physiology and historically the input from which that pattern was determined.

In another embodiment, the App may rely on obtaining its mood charting related inputs by recognizing patterns and trends detected in physiological inputs rather than those prompted by patient self-diagnosis. Long term self-diagnosis may ultimately allow for adequate information for patterns of physiology to take presence, allowing for this backwards/reverse implementation of obtaining mood charting related input to occur.

For an interactive mood charting algorithm, the method by which the input is requested will come in the form of an automatic message that requires patients to choose from a multiple choice questionnaire. This allows the questionnaire to give reference to specified time periods and allows the patients to input the information at their leisure.

There is a function to weigh each symptom within a set of symptoms—this is necessary for when severity alone doesn't capture a symptom's contribution to a state. For example, this may occur when the number and sets of symptoms are greater or less compared to previous states/episodes/cycles. Therefore, in this case, we must factor in not only the number of symptoms present but the weight attached to each symptom. Choosing the weight is a function of what symptom is causing the most influence. Or what are the main x-factors are causing instability. This doesn't necessarily coincide with what symptom is the most severe at that time. An example would be when two symptoms have the same severity but can differ in the presence of additional symptoms. In this case the psychiatrist would add more weight to the state with more symptoms. In mainstream psychiatry symptoms usually are viewed as part of a spectrum that range from less severe to most severe. To personalize the many combinations of symptoms and possible severities, weighing each factor would allows for individualized diagnosis, treatment and therapy and also for graphing a state to reflect a broader view with severity being captured in a multidimensional way that factors in the number of symptoms, severity of each symptom, and finally relevance of all of the above (weight).

Biological, psychological, and environmental factors may be key indicators in applying more weight to certain symptoms with less severity than other adjoining symptoms that are present at the same time. Thus, a weight established to acknowledge the level of impact that a symptom has on a person should come with documentation, which tells the patient/psychiatrist what X-factors are causing the most instability.

For example, in the presence of an environmental, biological, or psychological influence, a psychiatrist/patient may give more weight to anxiety or OCD than psychosis even though psychosis falls higher on the spectrum of severity. This allows the App to measure and document external influential factors, which may require intervention in order to therapeutically treat the range of symptoms that a patient is having.

In one embodiment, an interactive mood charting algorithm allows physiological correlations to be made by detecting patterns in the database. When the App detects a pattern within the sensor input, then any previous environmental, biological, and psychological factors will be referenced along with the other variables that are inquired about in interactive mood charting. Therefore these external factors may be documented upon detection and saved in the database for consideration as to how to weigh and scale symptoms that historically correlate to predetermined severities and weights. In regards to environmental factors, the App captures/stores the input and output of the interactive mood charting algorithm as reference to what symptoms require intervention. Also different therapies can target certain symptoms, while others remain untouched, thus weights would apply more heavily to the symptoms that couldn't be therapeutically changed.

There is a function to adjust the severity for these symptoms by a psychiatrist. This function may be used when the psychiatrist feels that there is an error in the self-diagnosis made by the patient. The psychiatrist uses both of these functions throughout the cycle. Along the way these functions must be able to collect and remember all similar factors that were present when the two functions were in effect.

In one embodiment, this invention includes multiple graphical representations of the timeline monitoring and output (in the form of sleep predicators, etc.) and other psychological and environmental variables for visual representation. This allows doctors to visually notice any significant trends. The application further includes statistical analysis tools that represent common baseline parameters between appointments, which allows psychiatrists to reach quick evaluation conclusions, without the error potential that would be present if making inferences from graphical representations.

Continuous monitoring of physiological processes coupled with modified mood charting and other psychological and environmental variables allows for adequate information that is required for visual/graphical representation.

In another embodiment, the present invention captures a probability estimation that can predict any deviations that may exist in a subjective input in absence of biomedical monitoring. Probability estimation is a function that is based on the assumption that history will more or less repeat itself in terms of symptoms and progression of severity from one cycle to the next, and that we are constantly updating like or probable conditions of the patient's previous cycle and stage of mania/depression. This information is used to estimate the variance between self-diagnosis and clinical diagnosis. Calculating the probability estimation involves several considerations, some of which include a psychiatrist's role.

It may be a psychiatrist's role to weigh each symptom in order to capture its relevant contribution to phases of mania and depression. This may in turn allow mania and depression to be accurately graphed.

A psychiatrist may adjust the severity based on clinical observations and how correlations/trends of the individual output compare to the already determined correlations/trends found in the database.

Any progression/digression of severities/weights based on trend analysis, should be a red flag requiring intervention. Upon analysis/communication severity and weight should be adjusted. Previous assumptions in the patent should aid the psychiatrist to adjust within reason. Notifications or red alerts may be patient specific according to the symptoms, medications, and external factors. The notification may come in many forms. An example could be forgetting to take medication, insomnia, or one of the baseline parameters that is based on statistical analysis of a patient's cycle. To the extent that these red alerts can be predicted and quantified, tools/functions should be provided as a means to apply to the input for alerts/notifications. Moreover, the study phase will provide opportunities to identify and quantify various factors that call for notifications, based upon evidence that can be identified in the input.

In the event that no alerts or notifications invite the psychiatrist to adjust severity and weight, then adjustments can be made upon patient's discretion. In the event that the majority of the inputs are stationary, then there should be an option to adjust severities and weights to the entire segments where rapid change doesn't occur.

For similar matches in the future, these functions (severity and weight) can recall the previous similar condition/state/episode and use them as an estimation, as to the rate of error that a patient holds with regards to self-diagnosis. Thus the patient's accuracy to self-diagnose their current symptoms can be guesstimated upon their ability to do so in the past under similar conditions of inputs that were identical and how their psychiatrist corrects their judgment for self-diagnosis.

Once this is done then the application calculates any/all deviations from the adjusted severity. This will then create a probability estimation that can be applied to a self-adjusting mood charting algorithm, in the absence of monitored readings. Furthermore, it will allow trends and severity of symptoms to be captured within a certain range that is dependent upon the user's ability to scale their own symptoms/side effects.

Specific consideration must be given to different stages in mania and depression. Mania and depression may be graphed as a bell curve. This allows a trend to capture a specified stage for a visual reference. Since the beginning and ending phases of mania and depression are difficult to self-diagnose, more probability should be considered to the respective tail ends of the curve.

Stages of mania/depression may be made by dividing the bell curve into four segments based on acceleration and deceleration of severity. Thus, we are comparing like symptoms, to like severities, to like segments in the stage of the given state. Also, use of the previous states/segments of the previous cycle (say last year's cycle) as an estimate as to how well a patient can self-diagnose themselves in reference to their previous abilities under similar conditions of their new cycle.

With each additional cycle the probability factor will decrease because patients may become better at self-diagnosis. With this being said, the system's of how well the patients can self-diagnose themselves should come from the most recent examples that are similar to the current cycle (the emphasis is on like factors because cycles/stages may slightly vary from previous ones).

Within each state multiple episodes are likely to occur, so individual analysis should be applied with separate consideration in terms of severity, frequency, and time elapsed. This is necessary for matching like episodes under the bell curve, even if time elapsed and progression of severity are seemingly uniform, specific episodes within may not fully match each other.

In order for psychiatrists to use this application, a smart-phone may prove to be inefficient. With the aid of monitored readings, psychiatrists need to document clinical observations against inputs to capture the probability estimation. For psychiatrist to receive and review data from collected from the patient, a larger screen is useful. For example, displaying monitored readings over a month would require a screen larger than a smart phone screen. Data stored in a central database may be viewed through a secure website. This website may contain real time updates of patient's progress. For accessibility of data in an organized manner, cloud computing may be used. That is, in some embodiments of the present invention, the inputs and/or outputs of the present system may be presented on a desktop, a tablet, a notebook, or any other computing device, not just a smartphone.

Another feature of the invention is that the server associated with the central database, upon the database recording specific symptoms or series of symptoms meeting pre-defined criteria, the server may intervene and send to the patient or medical professional an automatic text message and provide immediate steps to find the closest help. In another example, a recipient (psychiatrist) may receive an alert message in the form of a hyperlink. The hyperlink may be directed to a website for a relevant purpose, such as, for a third party conversation. Any web-related bilateral or multilateral communication may be performed using the hyperlink.

### Illustrative Inputs to the Present Invention

This listing is illustrative, and is not intended to be exhaustive:
Physiological: skin conductivity/body temperature, pulse, sleep (including the 4 phases of sleep and cycles of sleep per night, increased/decreased brain activity, physical activity level (measured by energy expended), blood pressure, vital signs, EEG, vocal chord vibration analysis, heart rate, muscle movements, EMG, respiration, UV light exposure
Mood: euphoric/elated, happy/content, poor frustration tolerance, irritable, overwhelmed, sad, loss of interest in normally pleasurable activities, hopeless, thoughts of suicide, persistent, unpleasant thoughts or worries, angry, rageful
Cognition: hyper focused, crystal clear thinking, fuzzy thinking, slowed thinking, rapid thinking/racing thoughts, chronic worries
Self-Perception: hopeful, brave/courageous, superior to others, sure of self/confident, decreased need for sleep, fearful, inferior to others, increased need for sleep
Energy: low, average, high
Behavior: impulsive, able to control behavior, motivated, organized/disorganized, violent, yelling/screaming, increased sex drive/risky sexual behaviors
Substances: alcohol, drugs (recreational, like medical marijuana, as well as prescription), caffeine, tobacco
Appetite: high, low, average, carb craving
Functioning: is the patient able to stay in normal/daily routine?
Exercise: none, activities and time spent doing the activities
Meditation: yes, no
Time spent outside today (in minutes and hours)
Environment: list daily stressors today, list pleasurable activities/occurrences today
Medications: name of medication, dose, frequency, supplements
Hydration: record glasses of water/juice, etc. (decaffeinated beverages only because caffeine is a diuretic and thus dehydrates)

In some embodiment, a parametric model may be situated in the present invention, and a machine learning algorithm may be applied for parameter estimation.

In one embodiment, physiology is monitored from wrist band devices, in a manner, such as, skin decreased to pH 7.5, or body temperature increased to 38° C.

For bipolar disorder, the key to the patient's learning their own patterns (to be able to predict patterns and avoid disease states) is sleep. In one embodiment, the sleep graph may be a top bar visually, and any other variables below for comparison. That way, they (patients) will begin over time to correlate mood, thinking, medication compliance, etc. with its effect on sleep. Mood is an important variable to compare with sleep visually, but any variable and multiple variables could visually be compared against sleep. Sleep change always precedes serious mood change.

Data collection over time has many advantages. Patients will begin over time to recognize their own personal triggers to the sleep changes that trigger disease. In one embodiment, once data is collected, graphing this out for patients on a separate screen of the App as a baseline would allow pull up of any particular day of concern to see how it correlates to their baseline, which would be useful in predicting insidious changes that cause disease. Individual intervention strategies for individual patients can be on the screen with the baseline data for reference and ease of use in prevention. The invention will learn more and more correlations over time and be able to re-rate the variables and signs and symptoms importance that are being monitored. This will generate valid research statistics and correlate them to real patient experiences.

Sleep information comes from the brain in the form of electrical signals in the brain. Electrical changes are energy changes that are going to accumulate to present as what we see clinically as a bipolar episode. Changing the sleep stops the momentum of the energy-change and starts a different energy-change into motion. If a sleep pattern is going to change, negatively affecting brain functions, then the earlier the intervention, the better, because a physics analogy dictates that energy in motion gains momentum—momentum is strength and power. A lot of momentum is difficult to reverse.

Symptoms can be early warning signs predicative of a change in sleep. Therefore, symptoms show up in patient's body before they fully manifest. However sleep is still the main X-factor because it alters the course of other symptoms/disease and is the main preventative measure, since correcting a sleep cycle can reverse other symptoms. It is not just the physiology that may change prior to a change in sleep cycle, but behavior may also change.

A self-adjusting algorithm of the present invention integrates multiple parameters to provide data and synthesis thereof, based on statistical probabilities from research, and serves as an adjunct to patients and clinicians in determining therapies, including pharmacologic and cognitive.

Patient centered program provides behavioral input and their choices. Clinical program provides clinicians with adjunctive information for clinical options. The present invention may be used as adjuncts in diagnosis, monitoring a therapy, determining therapeutic effects and efficacy. Therapy could be behavioral and pharmacologic.

The present invention integrates fixed values of disease, disease characteristics, lifestyle, and therapy with variable values of metrics from biosensors, and mood, emotional, and behavioral aspects in real time, and for medium and long term trends. Research identifies multi-parametric associations of fixed and variable factors that are significant, especially in pattern recognition. These associations are used to create algorithms that establish baseline for the individual (personalized baseline). Monitoring metrics over time produces raw changes from baseline, and algorithmic synthesis of changes (pattern recognition).

### CONCLUSIONS

One of ordinary skill in the art knows that the use cases, data flow, structures, hardware schematics, or flow diagrams may be performed in other orders or combinations, but the inventive concept of the present invention remains without departing from the broader spirit of the invention. Every embodiment may be unique, and methods/steps may be either shortened or lengthened, overlapped with the other activities, postponed, delayed, and continued after a time gap, such that every user is accommodated to practice the methods of the present invention.

The present invention may be implemented in hardware and/or in software. Many components of the system, for example, network interfaces, etc., have not been shown, so as not to obscure the present invention. However, one of ordinary skill in the art would appreciate that the system necessarily includes these components. A monitoring device is a hardware that includes at least one processor coupled to a memory. The processor may represent one or more processors (e.g., microprocessors), and the memory may represent random access memory (RAM) devices comprising a main storage of the hardware, as well as any supplemental levels of memory e.g., cache memories, non-volatile or back-up memories (e.g. programmable or flash memories), read-only memories, etc. In addition, the memory may be considered to include memory storage physically located elsewhere in the hardware, e.g. any cache memory in the processor, as well as any storage capacity used as a virtual memory, e.g., as stored on a mass storage device.

The hardware of a monitoring device also typically receives a number of inputs and outputs for communicating information externally. For interface with a user, the hardware may include one or more user input devices (e.g., a transceiver, Bluetooth communication, a keyboard, a mouse, a scanner, a microphone, a web camera, an interface for acquiring digital or analog signal, etc.) and a display (e.g., a Liquid Crystal Display (LCD) panel). For additional storage, the hardware my also include one or more mass storage devices, e.g., a floppy or other removable disk drive, a hard disk drive, a Direct Access Storage Device (DASD), an optical drive (e.g. a Compact Disk (CD) drive, a Digital Versatile Disk (DVD) drive, etc.) and/or a tape drive, among others. Furthermore, the hardware may include an interface with one or more networks (e.g., a local area network (LAN), a wide area network (WAN), a wireless network, and/or the Internet among others) to permit the communication of information with other computers coupled to the networks. It should be appreciated that the hardware typically includes suitable analog and/or digital interfaces to communicate with each other.

The hardware operates under the control of an operating system, and executes various computer software applications, components, programs, codes, libraries, objects, modules, etc. to perform the personalization techniques described above.

In general, the method executed to implement the embodiments of the invention, may be implemented as part of an operating system or a specific application, component, program, object, module or sequence of instructions referred to as “computer program(s)” or “computer code(s).” The computer programs typically comprise one or more instructions set at various times in various memory and storage devices in a computer, and that, when read and executed by one or more processors in a computer, cause the computer to perform operations necessary to execute elements involving the various aspects of the invention. Moreover, while the invention has been described in the context of fully functioning computers and computer systems, those skilled in the art will appreciate that the various embodiments of the invention are capable of being distributed as a program product in a variety of forms, and that the invention applies equally regardless of the particular type of machine or computer-readable media used to actually effect the distribution. Examples of computer-readable media include but are not limited to recordable type media such as volatile and non-volatile memory devices, floppy and other removable disks, hard disk drives, optical disks (e.g., Compact Disk Read-Only Memory (CD ROMS), Digital Versatile Disks, (DVDs), etc.), and digital and analog communication media.

Although the present invention has been described with reference to specific exemplary embodiments, it will be evident that the various modification and changes can be made to these embodiments without departing from the broader spirit of the invention. Accordingly, the specification and drawings are to be regarded in an illustrative sense rather than in a restrictive sense. It will also be apparent to the skilled artisan that the embodiments described above are specific examples of a single broader invention which may have greater scope than any of the singular descriptions taught. There may be many alterations made in the descriptions without departing from the spirit and scope of the present invention.

What is claimed is:

1. A method of creating a plurality of sleep predicators of a patient, said plurality of sleep predicators predicting a serious mood episode of said patient's chronic mental illness, the method comprising the steps of:
acquiring a physiological data and a behavioral data of said patient by using a means of signal acquisition, wherein said means of signal acquisition is selected from the group consisting of a transducer, a biomedical sensor, a surgically implanted sensor, a global positioning device, and a manually entered input;
forming a database of psychiatric records by recording said data in said database, wherein said database is coupled with a statistical engine having a sleep prediction algorithm, said engine adapted to compute sleep predicators, wherein said engine statistically computes a plurality of psychiatric records and said data, and wherein said plurality of psychiatric records are acquired via at least one of public medical records and private medical records;
mapping said acquired data in reference to said sleep prediction algorithm, in which the step of mapping comprises a statistical computation of said data, wherein said sleep prediction algorithm automatically adjusts based on an acquisition of a newer physiological data or a newer behavioral data, and wherein said sleep prediction algorithm achieves a statistical accuracy in sleep prediction by an iterative adjustment of said algorithm, wherein said iterative adjustment is based on said acquisition of said newer physiological or said newer behavioral data;
validating said sleep predictors from said sleep prediction algorithm by evaluating said physiological data or said behavioral data in relation to said sleep predictors, thereby forecasting that a sleep pattern in said patient is about to change; and
providing an output comprising said plurality of sleep predicators, wherein said output is displayed via a monitoring device, wherein said chronic mental illness is selected from the group consisting of mania, depression, bipolar disorder, schizophrenia, PTSD, and anxiety, and wherein said statistical computation comprises finding a first correlation between said acquired data and an illness symptom of said mental illness, wherein said illness symptom is statistically confirmed for accuracy by a first confirmation through said psychiatric records, thereby creating a therapeutic plan or a sleep remedy plan to prevent a further irreparable damage to said patient's mental state.

2. The method of claim 1, wherein said plurality of sleep predicators comprise a plurality of sleep analytics.

3. The method of claim 1, wherein said physiological data comprises at least one of vital signs, EEG, vocal cord vibration, temperature, heart rate, muscle movement, EMG, conductivity, resistance, respiration, and UV light exposure.

4. The method of claim 1, wherein said statistical computation comprises finding a second correlation between said acquired data and a therapeutic side effect on said mental illness, wherein said therapeutic side effect is relevant to a drug regimen of said patient, and wherein said therapeutic side effect is statistically confirmed for accuracy by a second confirmation through said psychiatric records.

5. The method of claim 1, wherein said means of signal acquisition further comprises at least one of electromechanical, optical, thermal, acoustic, and piezoelectric property.

6. The method of claim 1, wherein said manually entered input comprises a plurality of lifestyle related data, primarily comprising sleep, of said patient.

7. The method of claim 1, wherein said manually entered input comprises a plurality of mood related data of said patient.

8. The method of claim 1, wherein said output further comprises an analysis adjunctive to therapy.

9. The method of claim 1, wherein said output further comprises a cognitive analysis.

10. The method of claim 1, wherein said monitoring device comprises a portable device, a wearable device, an Internet device, or a cellular device.

11. The method of claim 1, wherein said means of signal acquisition is adapted to capture a probabilistic data of said patient.

12. A system for creating a plurality of sleep predicators of a patient, said plurality of sleep predicators predicting a serious mood episode of said patient's chronic mental illness, in a client-server environment, the system comprising:
a means of biomedical signal acquisition;
a monitoring device having a processor, a first memory, and a display;
a server having a second memory;
a database of psychiatric records linked to said server;
a communications-link between said monitoring device and said server; and
a plurality of computer codes embodied on said first memory and on said second memory, said plurality of computer codes which when executed, causes said means of biomedical signal acquisition, said device, and said server to respectively execute a process to:
acquire a physiological data and a behavioral data of said patient by using said means of biomedical signal acquisition, wherein said means of biomedical signal acquisition is selected from the group consisting of a transducer, a biomedical sensor, a surgically implanted sensor, a global positioning device, and a manually entered input;
form said database of psychiatric records by recording said data in said database, wherein said database is coupled with a statistical engine having a sleep prediction algorithm, said engine adapted to compute sleep predicators, wherein said engine statistically computes a plurality of psychiatric records and said data, and wherein said plurality of psychiatric records are acquired via at least one of public medical records and private medical records;
map said acquired data in reference to said sleep prediction algorithm, in which the step of mapping comprises a statistical computation of said data, wherein said sleep prediction algorithm automatically adjusts based on an acquisition of a newer physiological data or a newer behavioral data, and wherein said sleep prediction algorithm achieves a statistical accuracy in sleep prediction by an iterative adjustment of said algorithm, wherein said iterative adjustment is based on said acquisition of said newer physiological or said newer behavioral data;
validate said sleep predictors from said sleep prediction algorithm by evaluating said physiological data or said behavioral data in relation to said sleep predictors, thereby forecasting that a sleep pattern in said patient is about to change; and
provide an output comprising said plurality of sleep predicators, wherein said output is displayed via said monitoring device, wherein said chronic mental illness is selected from the group consisting of mania, depression, bipolar disorder, schizophrenia, PTSD and anxiety, and wherein said statistical computation comprises finding a first correlation between said acquired data and an illness symptom of said mental illness, wherein said illness symptom is statistically confirmed for accuracy by a first confirmation through said psychiatric records, thereby creating a therapeutic plan or a sleep remedy plan to prevent a further irreparable damage to said patient's mental state.

13. The system of claim 12, wherein said plurality of sleep predicators comprise a plurality of sleep analytics.

14. The system of claim 12, wherein said statistical computation comprises finding a second correlation between said acquired data and a therapeutic side effect on said mental illness, wherein said therapeutic side effect is relevant to a drug regimen of said patient, and wherein said therapeutic side effect is statistically confirmed for accuracy by a second confirmation through said psychiatric records.

15. The system of claim 12, wherein said output further comprises an analysis adjunctive to therapy.

16. The system of claim 12, wherein said monitoring device comprises a portable device, a wearable device, an Internet device, or a cellular device.

17. The system of claim 12, wherein said means of biomedical signal acquisition is adapted to capture a probabilistic data of said patient.

18. A monitoring device for monitoring a plurality of sleep predicators of a patient, said plurality of sleep predicators predicting a serious mood episode of said patient's chronic mental illness, the device capable of performing the steps of:
acquiring a physiological data and a behavioral data of said patient by using a means of signal acquisition, wherein said means of signal acquisition is selected from the group consisting of a transducer, a biomedical sensor, a surgically implanted sensor, a global positioning device, and a manually entered input;
forming a database of psychiatric records by recording said data in said database, wherein said database is coupled with a statistical engine having a sleep prediction algorithm, said engine adapted to compute sleep predicators, wherein said engine statistically computes a plurality of psychiatric records and said data, and wherein said plurality of psychiatric records are acquired via at least one of public medical records and private medical records;
mapping said acquired data in reference to said sleep prediction algorithm, in which the step of mapping comprises a statistical computation of said data, wherein said sleep prediction algorithm automatically adjusts based on an acquisition of a newer physiological data or a newer behavioral data, and wherein said sleep prediction algorithm achieves a statistical accuracy in sleep prediction by an iterative adjustment of said algorithm, wherein said iterative adjustment is based on said acquisition of said newer physiological or said newer behavioral data;
validating said sleep predictors from said sleep prediction algorithm by evaluating said physiological data or said behavioral data in relation to said sleep predictors, thereby forecasting that a sleep pattern in said patient is about to change; and
providing an output comprising said plurality of sleep predicators, wherein said output is displayed via said monitoring device, wherein said chronic mental illness is selected from the group consisting of a mania, a depression, a bipolar disorder, a schizophrenia, a PSDT and an anxiety, and wherein said statistical computation comprises finding a first correlation between said acquired data and an illness symptom of said mental illness, wherein said illness symptom is statistically confirmed for accuracy by a first confirmation through said psychiatric records, thereby creating a therapeutic plan or a sleep remedy plan to prevent a further irreparable damage to said patient's mental state.

19. The device of claim 18, wherein said device comprises a portable device, a wearable device, an Internet device, or a cellular device.

20. The device of claim 18, wherein said statistical computation comprises finding a second correlation between said acquired data and a therapeutic side effect on said mental illness, wherein said therapeutic side effect is relevant to a drug regimen of said patient, and wherein said therapeutic side effect is statistically confirmed for accuracy by a second confirmation through said psychiatric records.
